(19) United States
(12) Patent Application Publication
(10) Pub. No.: US 2001/0056074 A1
(43) Pub. Date: Dec. 27, 2001

(54) Novel lipophilic compounds having affinity with nucleic acids and therapeutical uses thereof

(76) Inventors: Jean-Jacques Yaouanc, Loc-Maria Plouzane (FR); Erwann Guenin, Brest (FR); Jean-Claude Clement, Coat-Meal (FR); Anne-Cecile Herve, Plouezoc'h (FR); Claude Ferec, Plougastel-Daoulas (FR); Virginie Floch, Brest (FR); Herve des Abbayes, Brest (FR)

Correspondence Address: Bierman, Muserlian and Lucas, 600 Third Avenue, New York, NY, 10016, US

(21) Appl. No.: 09/754,814

(22) Filed: Jan. 4, 2001

Related U.S. Application Data
(60) Provisional application No. 60/175,342, filed on Jan. 10, 2000.

(51) Int. Cl. A61K 48/00; C07J 51/00; C07F 9/70; C07F 9/28

(52) U.S. Cl. 514/44; 554/72; 554/78; 552/504; 552/506; 435/455

(57) ABSTRACT

The invention consists of a compound of the general formula (I) below:

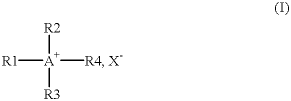

Wherein A, R1, R2, R3, R4 and X are as disclosed in the specification.

The invention also relates to the therapeutical uses of this compound, particularly for gene therapy.

### FIELD OF THE INVENTION

The invention lies in the field of compounds having affinity with nucleic acids and which may be used as non-viral vectors for introducing nucleic acids of interest within a desired host cell or a desired host organism.

### BACKGROUND OF THE INVENTION

There has been a great deal of interest in recent years in developing non-viral vectors for carrying DNA through cell membranes into the nuclei with a view to gene therapy. From the recent review of A. D. Miller on “cationic liposomes for gene therapy” (Angewandte Chem. Int. Ed. Engl., 1998, 37, 1768-1785), which presents a survey of described cationic lipids up to nowadays, it is striking to note that among all the cationic lipids described in prior art, their positive charge is always borne by a nitrogen atom.

Among the non-viral lipophilic compounds already known in the art are halides of −1,2 dioleoyl-3 trimethylammonium deoxyglycerol, commonly named DOTAP, of −1,2 dioleyl-3 trimethylammonium, commonly named DOTMA, of dimethylammonium ethyloxycarbonylcholesterol, commonly named DC-chol, and many phosphonolipids such as those described by G. Le Bolc'h et al. (Tetrahedron Lett., 1995, 36, 6681) or by V. Floch et al. (Eur. J. Med. Chem., 1998, 33, 12).

Nevertheless the poor transfection efficiency of the lipophilic non-viral vectors of prior art as well as their cell cytotoxicity properties has materialized a public need for non-viral vectors endowed with the same advantageous properties of the known compounds but which are endowed with increased transfection efficiency as well as with a lesser cytotoxicity.

### SUMMARY OF THE INVENTION

The inventors have now synthetized novel lipophilic compounds containing either a phosphonium or an arsonium cation and have shown that these compounds have a good affinity for nucleic acids and allow the introduction of a desired nucleic acid within a cell or a tissue of a host organism with a transfection efficiency which had never been observed using any one of the non-viral vectors described in prior art.

The inventors have surprisingly shown that the novel lipophilic compounds they have synthetized have much better properties than prior art lipophilic compounds which contain an ammonium cation.

Moreover, it has been shown according to the invention that the novel lipophilic compounds are useful for transfecting cells with a nucleic acid of interest both in vitro and in vivo and may then be used advantageously for gene therapy methods.

The present invention thus deals with these novel lipophilic compounds containing a phosphonium or an arsonium cation, that will be described in the detailed description of the invention. The lipophilic compounds of the invention contain a triallylphosphonium or a trialkyl arsonium cation as a polar head, with a counter-ion (anion), and a lipid moiety as well as a linker group that join the cation to the lipid moiety.

The invention also concerns vesicles comprising, or consisting mainly or almost exclusively of a lipophilic compound as described above.

Another object of the invention consists of a complex formed between a lipophilic compound as described above with a desired nucleic acid of interest.

The invention relates also with methods of gene therapy using a complex as described above, as well as with compositions, specifically pharmaceutical compositions, containing such a complex and usable when performing a method of gene therapy.

### BRIEF DESCRIPTION OF THE FIGURES

FIG. 1 illustrates a scheme for the synthesis of a lipophilic compound according to the invention and having the formula (V) and wherein R6 is a methyl group.

FIG. 2 illustrates a scheme showing the synthesis of a lipophilic compound according to the invention and having the formula (V) and wherein R6 is an ethyl group.

FIG. 3 illustrates a scheme showing the synthesis of a lipophilic compound according to the invention and having the formula (V) and wherein R6 is a propyl group.

FIG. 4 illustrates a scheme showing the synthesis of different lipophilic compounds according to the invention.

The upper line shows the synthesis of a lipophilic compound wherein R1 is of formula (II);

The middle line shows the synthesis of a lipophilic compound according to the invention and wherein R¹ has the formula (III);

The bottom line shows the synthesis of a lipophilic compound according to the invention and wherein R1 has the formula (IV).

FIG. 5 illustrates the transfection efficiency of different lipophilic compounds according to the invention wherein R1 has the formula (V).

Cells of respectively K 562 and HeLa cell lines have been transfected with pTG 11033 plasmid encoding luciferase protein that has been previously complexed with a lipophilic compound, either:

a lipophilic compound wherein A is a nitrogen atom; or

a lipophilic compound of the invention wherein A is a phosphorus atom; or

a lipophilic compound of the invention wherein A means an arsonium atom.

The ordinates represent the total relative light units corresponding to the light emitted by the luciferase protein synthesised in the transfected cells, which reflects the amount of protein produced and thus the efficiency of the transfection performed.

FIG. 6 illustrates the cell cytotoxicity on K 562 cell line of respectively:

a lipophilic compound containing a nitrogen atom (grey bar at the bottom of FIG. 6);

a lipophilic compound of the invention wherein A means an arsonium atom; and

a lipophilic compound of the invention wherein A means a phosphorus atom.

The abscissa represent the number of viable cells counted at a time of 48 hours after cell transfection.

FIG. 7 illustrates the transfection efficiency on CFT1, K 562 and HeLa cell lines with a lipophilic compound containing a nitrogen atom and wherein R6 means a methyl group (n=1), an ethyl group (n=2) or a propyl group (n=3).

The ordinates represent the value of the total relative light units obtained for 16 different wells.

FIG. 8 illustrates the transfection efficiency on CFT1, K 562 and HeLa cell lines with a lipophilic compound wherein R1 is of formula V, containing a phosphorus atom and wherein R6 means a methyl group (n=1), an ethyl group (n=2) or a propyl group (n=3).

FIG. 9 illustrates the transfection efficiency on CFT1, K 562 and HeLa cell lines with a lipophilic compound wherein R1 is of formula V, containing an arsonium atom and wherein R6 means a methyl group (n=1), an ethyl group (n=2) or a propyl group (n=3).

FIG. 10 illustrates the transfection efficiency of different lipophilic compounds containing different kinds of fatty acid chains, respectively C₁₄:0 and C₁₈:1.

The group of bars on the left reflects the transfection efficiency with a lipophilic compound containing a nitrogen atom;

the middle group of bars reflects the transfection efficiency with a lipophilic compound of the invention wherein R1 is of formula V and containing a phosphorus atom; and

the group of bars on the right reflects the transfection efficiency with a lipophilic compound of the invention wherein R1 is of formula V and containing an arsonium atom.

FIG. 11 illustrates the transfection efficiency of different lipophilic compounds containing different kinds of fatty acid chains, respectively C₁₄:0 and C₁₈:1.

FIG. 12 illustrates a comparison between lipophilic compounds containing nitrogen atom and lipophilic compounds according to the invention, as regards both to their respective transfection efficiency and their cell cytotoxicity.

The bars reflect the transfection efficiency which is measured as the total relative light units obtained for 16 wells (TRLU).

Each dot of the line reflects the cell cytotoxicity as measured by the toxicity index which is measured as described in the Materials and Methods section.

FIG. 13 illustrates the respective transfection efficiency of:

a lipophilic compound which contains a nitrogen atom;

a lipophilic compound of the invention wherein R1 has the formula (III) and which contains a phosphorus atom; and

a lipophilic compound of the invention wherein R1 has the formula (III) and which contains an arsonium atom.

FIG. 14 illustrates the in vivo transfection efficiency respectively of a lipophilic compound containing a nitrogen atom and different lipophilic compounds of the invention wherein R1 is of formula V containing either a phosphorus atom or an arsonium atom and two different lipid moieties.

### DETAILED DESCRIPTION OF THE INVENTION

The present inventors have thus attempted to obtain novel compounds endowed with a high transfection efficiency together with a decrease in cell toxicity.

They have surprisingly found that phosphonium and arsonium cationic lipids have much better transfection properties and much lesser cytotoxic side effects than the lipophilic compounds already known in the art.

Thus, a first object of the invention consists of a compound of the general formula (I) below:

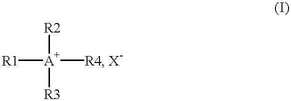

Wherein A is a phosphorus or an arsenic atom; X⁻ is an anion; and

wherein R1 is selected from the group consisting of:

a) the radical of formula (II) below:

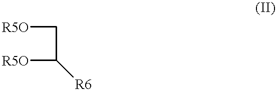

wherein R5 represents a lipid moiety and R6 is a linear or branched alkyl chain from 1 to 4 carbon atoms.

Provided that R2, R3 and R4 of formula (I) represent each a methyl group;

b) the radical of formula (III) below:

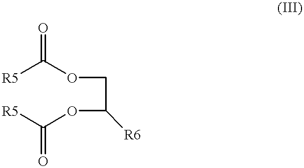

wherein R5 represents a lipid moiety and R6 is a linear or branched alkyl chain from 1 to 4 carbon atoms,

provided that R2, R3 and R4 of formula (I) represent each a methyl group;

c) the radical of formula (IV) below:

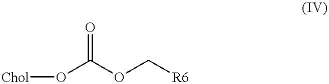

wherein Chol means a cholesteryl radical and R6 is a linear or branched alkyl chain from 1 to 4 carbon atoms,

provided that R2, R3 and R4 of formula (I) represent each a methyl group; and

d) the radical of formula (V) below:

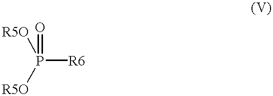

wherein R5 represents a lipid moiety and R6 is a linear or branched alkyl chain from 1 to 4 carbon atoms,

provided that R2 and R4 are alkyl chains from 1 to 4 carbon atoms; and

R3 is selected from the group consisting of:

an alkyl chain as defined for R2 and R4;

the functional group CH₂—CH₂—P⁺ (R6R7R8), wherein R6, R7 and R8 have the same meaning as R2 and R4; and

—CH₂—CO₂R9 wherein R9 has the same meaning as R2.

The inventors have shown in the Examples 9 and 10 below that the lipophilic compounds of the invention as described above allow a high transfection efficiency of nucleic acids within host cells, as compared to analogues containing an ammonium cation, and that they are much less cytotoxic than lipophilic compounds containing an ammonium cation.

They have also shown that the transfection property of a compound according to the invention was increased when the group R6 as defined above was an ethyl or a propyl group, as compared when R6 means a methyl group. This is particularly obvious for compounds wherein R1 is of the Formula (V), as shown in Example 9, Section 2.

Moreover, it has also been shown that the transfection efficiency was increased when the R5 lipid moiety means an alkenyl chain, though good transfection efficiency was obtained when the R5 lipid moiety is an alkyl chain, and more particularly a linear alkyl chain, such as a linear alkyl chain of 14 carbon atoms. This is particularly observed for lipophilic compounds of the invention wherein R1 is of the Formula (V), as described in Example 9, Section 3.

It has also been shown by the inventors that the lipophilic compounds of the invention were endowed with both a high transfection efficiency and a low cytotoxicity, whereas lipophilic compounds containing a nitrogen atom as the cation were in contrast both more cytotoxic and less efficient for transfection, for the same amounts of compounds used. These results are particularly illustrated in Example 9, Section 4.

An another essential property of a lipophilic compound according to the invention is that it is equally efficient for in vitro as well as for in vivo transfection of nucleic acids, as demonstrated particularly in Example 10 below.

In a first embodiment, the compound of the invention as defined above is such that the anion X is selected from the group consisting of an halide, CF₃SO₃⁻, CF₃CO₂⁻ or HSO₄⁻.

Preferably, the halide is selected from the group consisting of Cl⁻, Br⁻ and I⁻.

In a second embodiment, the compound of the invention as defined above is such that the R5 lipid moiety is selected from the group consisting of:

(i) an alkyl or an alkenyl chain containing from 10 to 22 carbon atoms comprising 0, 1 or 2 olefinic double bonds,

(ii) a cholesteryl derivative

(iii) a perfluoro alkyl chain from 10 to 22 carbon atoms.

Preferably the R5 lipid moiety is selected from the group consisting of C_{14 0}, C_{18 1}, C_{18 2}; C_{15 0}, C_{17.0}, C_{17.1}, C_{17 2}, wherein the first number designates the number of carbon atoms and the second number designates the number of double bonds.

In a third embodiment, the compound of the invention as defined above is such that when R1 is of formula (V), R2 and R4 represent each independently a radical selected from the group consisting of CH₃, C₂H₅, nC₃H₇, iso C₃H₇, with n being an integer equal to 1, 2 or 3.

A first group of preferred lipophilic compounds according to the invention are those wherein R1 has the formula (II), (III) or (V), the R5 lipid moiety consists of an alkyl chain and R6 is a methyl group.

A second group of preferred lipophilic compounds according to the invention are those wherein R1 has the formula (II), (III) or (V), the R5 lipid moiety consists of an alkenyl chain and R6 is a methyl group.

A third group of preferred lipophilic compounds according to the invention are those wherein R1 has the formula (II), (III) or (V), the R5 lipid moiety consists of an alkyl chain and R6 is an ethyl group.

A fourth group of preferred lipophilic compounds according to the invention are those wherein R1 has the formula (II), (III) or (V), the R5 lipid moiety consists of an alkenyl chain and R6 is an ethyl group.

A fifth group of preferred lipophilic compounds according to the invention are those wherein R1 has the formula (II), (III) or (V), the R5 lipid moiety consists of an alkyl chain and R6 is a propyl group.

A sixth group of preferred lipophilic compounds according to the invention are those wherein R1 has the formula (II), (III) or (V), the R5 lipid moiety consists of an alkenyl chain and R6 is a propyl group.

A seventh group of preferred lipophilic compounds according to the invention are those wherein R1 has the formula (II), (III) or (V), the R5 lipid moiety consists of a cholesteryl —[C(O)N—CH₂—CH₂—O)] group and R6 is an ethyl group.

A eighth group of preferred lipophilic compounds according to the invention are those wherein R1 has the formula (II), (III) or (V), the R5 lipid moiety consists of a perfluoroalkyl chain R6 is an ethyl group.

A ninth group of preferred lipophilic compounds according to the invention are those wherein R1 has the formula (II), (III) or (V), the R5 lipid moiety consists of an oleoyl chain (C₁₇H₃₃C(O)O) and R6 is a propylene group.

A tenth group of preferred lipophilic compounds according to the invention are those wherein R1 has the formula (II), (III) or (V), the R5 lipid moiety consists of an oleyl chain (C₁₈H₃₅) and R6 is a −1,2 deoxyglycerol group.

A eleventh group of preferred lipophilic compounds according to the invention are those wherein R1 has the formula (II), (III) or (V), the R5 lipid moiety consists of a cholesteryl group and R6 is a [C(O)O—CH₂—CH₂—] group.

The phosphonium and arsonium derivatives of phosphonolipids wherein R1 is of formula (V) are synthesized by different methods, depending on the chain length between the phosphonate and the cation:

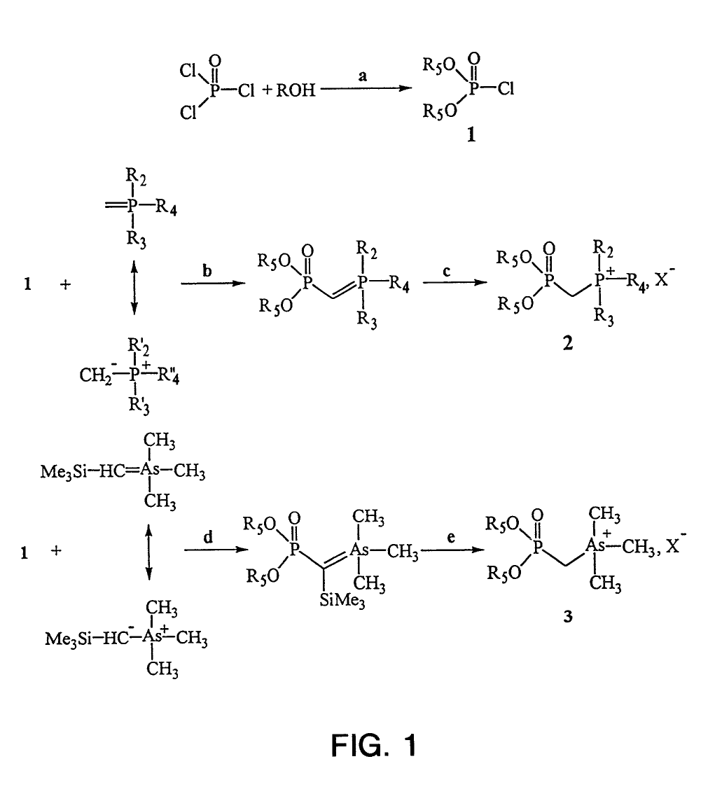

a) The synthesis of compounds where n=1 was achieved according to the scheme presented in FIG. 1: the addition of fatty chlorophosphates 1 on phosphorus or arsenic ylides lead to phosphonium and arsonium phosphonolipids 2 and 3. For arsenic, the silylated ylids are more convenient. After addition, the silyl group is removed by methanol or water or trimethylsilanol.

In FIG. 1, the synthesis conditions for the compounds wherein R5=C₁₄H₂₉, C₁₈H₃₅; R2, R3 and R4=alkyl ; X=Halogen are the followings:

a) Et₂O, NEtiPr₂ (4 eq) 72 h, 20° C.

b) THF, 1 h, 0° C.

c) THF, HX, 1 h, 20° C.; H₂O/NaX, 24 h 20° C.

d) Et₂O, 1 h, 0° C.

e) Et₂O, HX, 1 h, 20° C. Et₂O, Me₃SiOH, MeOH; or H₂O/NaX, 24 h, 20° C.

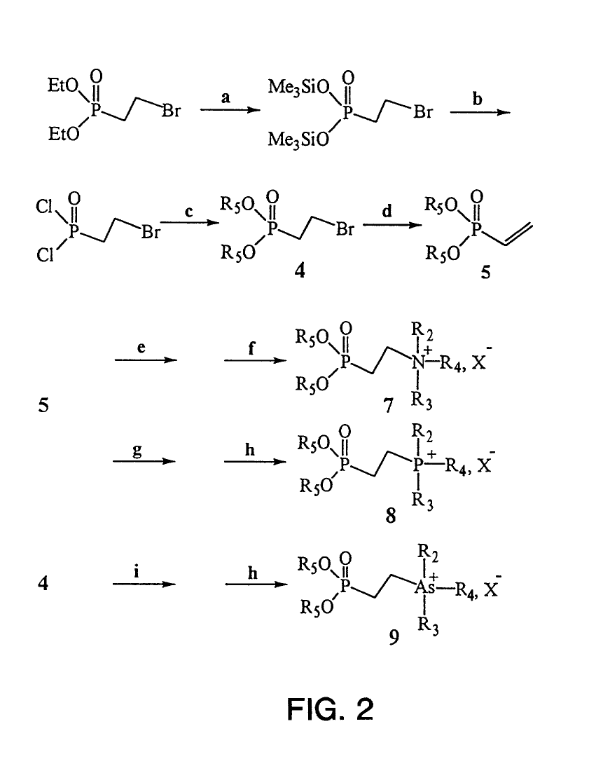

b) Synthesis of compounds of the invention wherein n=2 are illustrated in FIG. 2. The phosphonium salts were synthesized by addition of acidic H phosphonium salts on fatty vinyl phosphonate 5, whereas the corresponding arsonium salts resulted from direct quaternarization of trialkyl arsines by fatty 2-bromoethylphosphonates (FIG. 2).

In FIG. 2, the synthesis conditions for the compounds wherein R5=C₁₄H₂₉, C₁₈H₃₅, C₁₈H₃₃; R2, R3 and R4=alkyl groups; X=Halogen, trifluoromethanesulfonate, hydrogen sulfate, are the followings:

a) CH₂Cl₂, BrSiMe₃, 24 h, 20° C.

b) CH₂Cl₂, Oxalylchloride, DMF, 2 h, 20° C.

c) Et₂O, Alcohol, EtNiPr₂, 24 h, 20° C.

d) THF, NEt₃, 48 h, reflux.

e) EtOH, HNR′₂, 96 h, 20° C.;

f) Et₂O, R″X, 24 h, 20° C.;

g) DMF, [HR″R′₂P⁺, X⁻], 48 h, reflux;

h) CH₂Cl₂, NaX/H₂O, 48 h, 20° C.;

i) Sealed tube, AsR₃, 72 h, 70° C.;

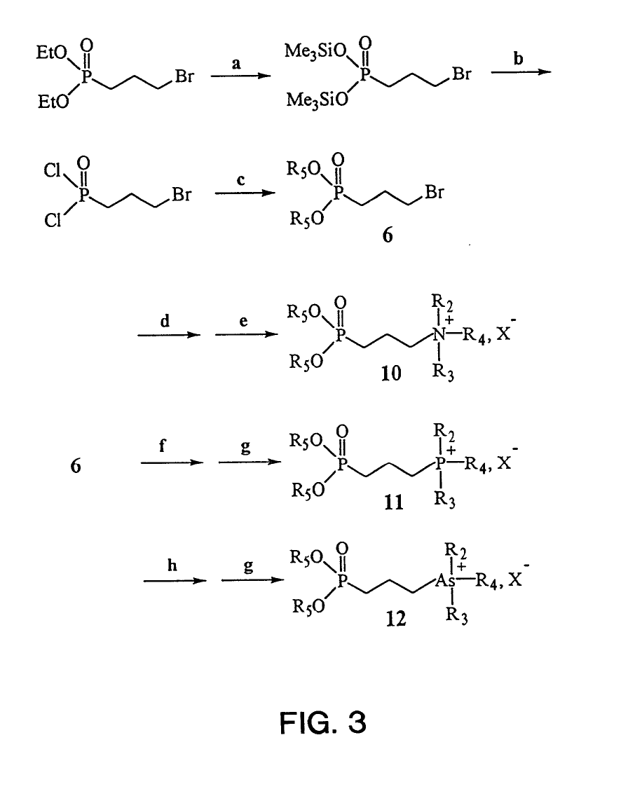

c) The synthesis of compounds according to the invention wherein n=3, is illustrated in FIG. 3. In both cases, phosphonium and arsonium halides resulted from direct quaternarization of trialkylphosphines and trialkylarsines by fatty 3-bromopropylphosphonates 6 (FIG. 3). Starting fatty phosphites and phosphonates were prepared by adapting known procedures of the literature.

In FIG. 3, the synthesis conditions for the compounds wherein R5=C₁₄H₂₉, C₁₈H₃₅, C₁₈H₃₃; R2, R3 and R4=alkyl groups; X=Halogen, trifluoromethanesulfonate, hydrogen sulfate are the followings:

Steps a), b) and c) are equal to Scheme 2 (FIG. 2), and

d) EtOH, HNR′₂, 96 h, 20° C.

e) Et₂O, R″X, 24 h, 20° C.

f) THF, PR′₃, 24 h, 20° C.

g) CH₂Cl₂, NaX/H₂O, 48 h, 20° C.

h) Sealed tube, AsR₃, 72 h, 70° C.

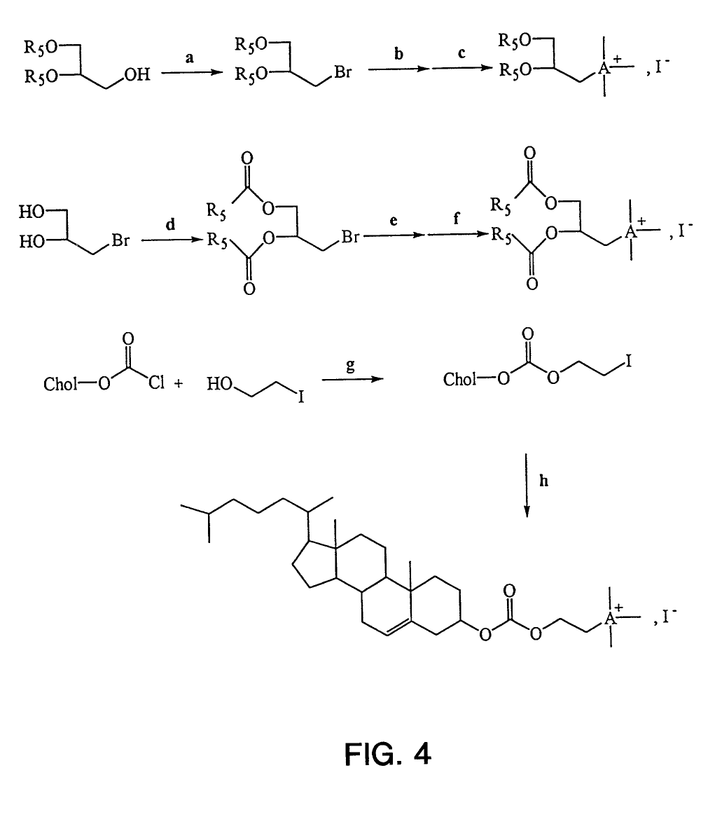

Besides the fatty cationic phosphonates above, were also synthesized phosphonium and arsonium analogues of known cationic lipids containing an ammonium polar head, such as DOTMA (P. L. Felgner et al., Proc. Natl.Acad. Sci. USA, (1987), 84, 7413-7417), or DOTAP (H. Abken et al., ibid, (1993), 90, 6518), or DC Chol (X. Gao and L. Huang, Biochem. Biophys. Res. Commun., (1991), 179, 280-285), according to the scheme of FIG. 4, and wherein R1 is of formula (II), (III) and (IV), respectively.

In FIG. 4, the synthesis conditions for the compounds wherein R1 is of formula (II′) (upper line), (III) (middle line) or (IV) (bottom line) and wherein A is a phosphorus or an arsenic atom; R5=C₁₈H₃₅ R2, R3 and R4=C₁₅H₃₁, C₁₇H₃₅ and C₁₇H₃₃, are the followings:

a) THF, PBr₃, 3 h, 20° C.

b) Sealed tube, A(Me)₃, one week, 40° C.

c) CH₂Cl₂, NaX/H₂O, 48 h, 20° C.

d) THF, R′COOH, DCC, DMAP, 3 h, 20° C.

e) THF, Nal, reflux.

f) Sealed tube, A(Me)₃, one week, 40° C.

g) CH₂Cl₂, DMAP, 72 h, 25° C.

h) Sealed tube, A(Me)₃, 72 h, 40° C.

Any one of the lipophilic compounds described above may be used as such in solution for complexing with a nucleic acid, the introduction of which in a cell host or in a host organism is sought.

Without willing to be bound by any particular theory, the inventors believe that the portion of the molecules that bears the phosphonium or the arsonium cation has the ionic properties of a lipophilic compound of the invention that confers its affinity for nucleic acids and thus represent the “carrier” portion of the molecule, whereas the lipid moiety allows the lipophilic compound to bind to cell membranes, goes through the cell membrane lipid bilayer and then reaches the cytoplasm and/or the nucleus wherein the nucleic acids of interest are transcribed or alternatively hybridize with a desired target nucleic acid naturally present within the cell.

As used interchangeably herein, the terms “nucleic acid”, “polynucleotide” and “oligonulcleotide” include DNA, RNA, DNA/RNA hybrid sequences of more than two nucleotides in length in either single chain or duplex form.

In one preferred embodiment, a lipophilic compound according to the invention is processed so as to obtain vesicles before incubating the thus obtained vesicles with a nucleic acid of interest in order to form a complex between said lipophilic compound and said nucleic acid of interest.

Preferably, the vesicle prepared consists essentially of a lipophilic compound according to the invention.

Preferably, the vesicle prepared consists of a small unilamellar vesicle.

In another preferred embodiment, the vesicle consists of a multilamellar vesicle.

In one embodiment, the nucleic acid to be introduced into a cell encodes a protein or a peptide. The protein can be any protein useful in gene therapy, including, but not limited to, cytokines, structural proteins, antigens, immunogens, receptors, transcription factors.

In another embodiment, the nucleic acid encodes an antisense polynucleotide which hybridizes with a desired target nucleic acid sequence, the inhibition of the expression of which target nucleic acid is sought.

Expression of antisense nucleic acids can be used, e.g., to reduce or inhibit translation of a mRNA into a specific protein.

In yet another embodiment, the nucleic acid to be transcribed is a reporter gene. Reporter genes include any gene encoding a protein, the amount of which can be determined. Preferred reporter genes include the luciferase gene, the β-galactosidase gene (LacZ), the chloramphenicol acetyl transferase (CAT) gene, or any gene encoding a protein providing resistance to a specific drug.

The nucleic acid to be introduced into a cell or administered within an animal organism may be operably linked to a regulatory sequence.

Preferably, the regulatory sequence is selected so as to be functional within the cell host or within the host animal organism wherein the expression of the nucleic acid is sought.

The nucleic acid which is to be introduced into a cell or to be administered to a animal organism and which is complexed with a compound according to the invention may be a double stranded or a single stranded nucleic acid which can be either linear or circular.

In a specific embodiment, said nucleic acid is a recombinant vector, preferably an expression vector wherein the nucleic acid to be expressed is operably linked to a suitable regulatory sequence.

Most preferably, the expression vectors are used for in vivo or in vitro transfection and expression of genes in particular cell types (e.g. muscle, skin, liver etc.).

According to the invention, the linear or circular nucleic acid of interest is firstly complexed with a compound according to the invention before introducing the complex into the desired cell host or animal organism.

In one specific embodiment, the nucleic acid of interest is incubated with a compound of the invention which has previously been prepared under the form of vesicles, preferably under the form of unilamellar vesicles.

Like most non viral methods of gene transfer already known by the one skilled in the art, the use of a non viral method of gene transfer according to the invention by using a complex between a nucleic acid of interest and a lipophilic compound of the invention rely on normal mechanisms used by animal cells, preferably mammalian cells, for the uptake and intracellular transport of macromolecules.

In preferred embodiments, non-viral targeting means of the present invention rely on endocytic pathways for the uptake of genes by the targeted cell.

Thus, the invention also relates to a method for introducing in vitro a nucleic acid in a cell host comprising the steps of:

a) incubating said nucleic acid with a compound according to the invention to obtain complexes formed between said nucleic acid and said compound; and

b) incubating the cell host with the complexes obtained at step a).

In a specific embodiment of the method above, the compound having affinity for nucleic acid of the invention is under the form of multilamellar or unilamellar vesicles, preferably small unilamellar vesicles.

As already mentioned, the complexes between a compound of the invention and a nucleic acid of interest may be delivered by any method that delivers injectable materials to the cells of an animal, such as, injection into the interstitial space of tissues (heart, muscle, skin, lung, leaver, intestine and the like). These polynucleotides constructs complexed with a compound of the invention can be delivered in a pharmaceutically acceptable liquid or aqueous carrier.

The complexes between a compound of the invention and a nucleic acid of interest used in a gene therapy method are preferably constructs that will not integrate into the host genome nor will they contain sequences that allow for replication. Any strong promoter known to those skilled in the art can be used for driving the expression of the nucleic acid of interest. Unlike other gene therapies techniques, one major advantage of introducing a complex between a compound of the invention and the nucleic acid of interest into target cells is the transitory nature of the polynucleotide synthesis in the cells. Studies have shown that non replicable DNA sequences can be introduced into cells to provide production of the desired polypeptide for periods of up to six months.

The complex between a compound of the invention and a nucleic acid of interest can be delivered to the interstitial space of tissues within the animal, including of muscle, skin, brain, lung, liver, spleen, marrow, thymus, heart, blood, bone, cartilage, pancreas, kidney, stomach, intestine, testis, ovary, uterus, rectum, nervous system, eye, gland and connective tissue.

Interstitial space of the tissues comprises the intracellular fluid, mucopolysaccharide matrix among the reticular fibres of organ tissues, elastic fibres in the walls of vessels or chambers, collagen fibres or fibrous tissues, or that same matrix within connective tissue unsheathing muscle cells or the lacunae of bone.

Delivery to the interstitial space of muscle tissue is preferred because it has been widely shown in the art that a specific DNA sequence may be expressed in muscle tissue during a long period of time due to the stability and the low speed regeneration of the muscle tissue, and that the transcription and the translation products may then circulate systematically due to the high vascularisation of the muscle tissue.

The complexes between a compound of the invention and a nucleic acid of interest may be conveniently delivered by injection into the tissues comprising the targeted cells. They are preferably delivered to and expressed in persistent non dividing cells which are differentiated, although delivery and expression may be achieved in non differentiated or less completed differentiated cells, such as, for example, stem cells of blood or skin fibroblasts.

For injection of a complex between a compound of the invention and a nucleic acid of interest, an effective dosage amount of DNA or RNA will be in the range of from about 0,005 mg/kg body weight to about 50 mg/kg body weight. Preferably, the dosage will be from about 0,005 mg/kg to about 20 mg/kg and more preferably from about 0.05 mg/kg to about 5 mg/kg body weight.

Of course, as the one skilled in the art will appreciate, this dosage will vary according to the tissue site of injection.

The appropriate and effective dosage of nucleic acid sequence can readily be determined by those of ordinary skilled in the art and may depend on the condition being treated and the route of administration.

The preferred route of administration is by the parenteral route of injection into the interstitious space of tissues. However, other parenteral routes may be also be used, such as, inhalation of an aerosol formulation particularly for delivery to lungs or bronchial tissues, throat or mucous membranes of the nose.

In another preferred embodiment, a complex between a compound of the invention and a nucleic acid of interest can be delivered by intravenous injection, including a delivery to arteries during angioplasty by the catheter used in the procedure.

Thus, the invention also concerns a method for introducing in vivo a nucleic acid in cells of an host organism comprising the steps of:

a) incubating said nucleic acid with a compound according to the invention to obtain complexes formed between said nucleic acid and said compound; and

b) administering the complexes obtained at step a) to said host organism.

As already mentioned, a preferred organism is an animal and most preferably a mammal.

The invention also relates to a complex formed between a nucleic acid and a compound according to the invention.

Usually, such a complex between a compound of the invention and a nucleic acid of interest is obtained by placing a compound of the invention is a suitable solution such as sterile pyrogen-free distilled water, and then adding to the solution containing the compound of the invention a suitable volume of sterile pyrogen-free distilled water containing the desired amount of the nucleic acid of interest and incubating the compound of the invention and the nucleic acid of interest for a desired period of time which is preferably comprised between about 15 min and one hour and most preferably between about 20 min and 45 min. Then, the complexes are formed and can be administered to the desired cell or to the animals for respectively in vitro and in vivo transfections.

Resulting formulation may be used as such or stabilized with adjuvants such as tween® (20, 40, 60 or 80), NaCl or DMPE-PEG 5000.

In one specific embodiment, the compound of the invention is firstly obtained under vesicles, preferably small unilamellar vesicles that are prepared by sonicating a compound of the invention for the desired period of times, such as for example between 5 and 20 minutes and preferably for about 10 minutes in the chamber of a sonicator apparatus.

Techniques of preparing lipid vesicles by sonicating lipophilic compounds are well known from the one skilled in the art.

As already described above, the nucleic acid may comprise a polynucleotide encoding a polypeptide of interest, and specifically a polypeptide of therapeutical interest.

In another embodiment, the nucleic acid of interest encodes an antisense polynucleotide that hybridizes to a targeted nucleic acid, the inhibition of the expression of which is sought.

In a most preferred embodiment, the polynucleotide encoding a polypeptide or an antisense polynucleotide is operably linked to a regulatory sequence, most preferably a regulatory sequence which is functional within the cell or within the animal organism in which the expression of the polynucleotide encoding the polypeptide or the antisense polynucleotide is sought.

The invention further relates to a composition comprising a complex formed between a compound of the invention and a nucleic acid of interest.

A further object of the invention consists of a pharmaceutical composition comprising a complex formed between a compound of the invention and a nucleic acid of interest.

Such a pharmaceutical composition to be administered to the desired host organism contains an amount of the nucleic acid of interest that varies according to the site of injection. As an indicative dose, it will be injected between 0,005 mg/kg and 50 mg/kg body weight of the nucleic acid of interest under the form of a complex with a compound of the invention, in an animal body, preferably a mammal body, for example a mouse body.

A complex between the compound of the invention and a nucleic acid of interest comprises preferably a ratio between the amount of a compound according to the invention and the amount of the nucleic acid of interest which is comprised between 1 and 10, and more preferably 2 and 6, in weight.

The invention now being generally described, it will be more readily understood by reference to the following examples which are included merely for purposes of illustration of certain aspects and embodiments of the present invention, and are not intended to limit the invention.

### EXAMPLES

### Example 1

### Synthesis of [ditetradecyl(trimethylphosphoniomethyl)phosphonate]iodide

As a preliminary remark, the structure of each new compound described in examples 1 to 8 was clearly ascertained by ¹H, ¹³C and ³¹P NMR spectroscopy.

To a three necked flask was added 2.2 grams of tetramethylphosphonium iodide, 10 mL of dry tetrahydrofuran (Aldrich) and 4 mL of butyllithium (2.5 M hexane solution, Aldrich,). The reaction mixture was stirred half an hour at room temperature, then added dropwise, at −10 degree C., to a three necked flask containing 2.5 grams of chloroditetradecylphosphate dissolved in 10 mL of dry tetrahydrofuran (Aldrich). The reaction mixture was stirred for three hours at room temperature. An hydrochloric solution (1 M in ether, Aldrich) was then added till acidic pH. Organic phase was washed by 20 mL of water and evaporated under vacuum. The residue was made soluble in 10 mL of dichloromethane, and 5 mL of an aqueous solution saturated by sodium iodide was added. After 24 hours stirring at room temperature organic phase was separated, dried under MgSO₄ and evaporated under vacuum. The product was precipitated from a dry ether solution at −4 degree C. (yield: 75%).

### Example 2

### Synthesis of [ditetradecyl (trimethylarsoniomethyl)phosphonate]iodide

Synthesis protocols are equal to trimethylphosphoniophosphonate. The trimethylsilylated arsonium ylide was made from addition of 4 mL of a butyllithium solution to 3.2 grams of trimethyl(trimethylsilylmethyl)arsonium iodide (yield 65%).

### Example 3

### Synthesis of [ditetradecyl(2-trimethylphosphonioethyl)phosphonate]iodide

2.3 grams of acidic trimethylphosphonium chloride and 5.0 grams of ditetradecylvinylphosphonate were heated at the reflux of 10 mL of dry dimethylformamide, for 72 hours, under nitrogen. The solvent was then evaporated under vacuum. Product was taken up in dichloromethane and washed twice by 10 mL of water, then organic phase was dried on MgSO₄ and evaporated. Counter-ion was changed by metathesis with a mixture dichloromethane/aqueous solution saturated with sodium iodide. After treatment (washings and evaporation) the product is purified by successive precipitation in diethylether at −4 degree C., and isolated as a white powder (yield: 75%).

### Example 4

### Synthesis of [ditetradecyl(2-trimethylarsonioethyl)phosphonate]iodide

2.9 grams of ditetradecyl(2-bromoethyl)phosphonate and 0.9 mL of trimethylarsine were heated in sealed tube (closed under vacuum), for a week, at 45 degree C. The product was then taken up in dichloromethane and washed twice by 10 mL of water. Metathesis of the counter-ion was done and after treatment (washings, drying, evaporation) the product was purified by successive precipitation in diethylether (yield 90%).

### Example 5

### Synthesis of ditetradecyl(3-trimethylphosphoniopropyl)phosphonate iodide

10 mL of a trimethylphosphonium solution (1M solution in tetrahydrofuran) and 2.9 grams of ditetradecyl(3-bromopropyl)phosphonate were reacted, in 10 mL tetrahydrofuran, for 3 days at reflux. After evaporation, the product was taken up in 15 mL of dichloromethane, washed by 10 mL of hydrochloric acid (10% w/w). Organic phase was extract and 5 mL of an aqueous solution saturated by sodium iodide was added. The mixture was stirred, at room temperature, for 24 hours. Organic phase was extracted, washed by twice 10 mL of water, dried and evaporated under vacuum. The product was purified by successive precipitation in diethylether at −4 degree C. and isolated as a white powder (yield: 90%).

### Example 6

### Synthesis of ditetradecyl(3-trimethylphosphoniopropyl)phosphonate iodide

10 mL of a trimethylphosphine solution (1M solution in tetrahydrofuran) and 2.9 grams of ditetradecyl(3-bromopropyl)phosphonate were heated in 10 mL tetrahydrofuran for 3 days at reflux. After evaporation, the crude product was taken up in 15 mL of dichloromethane, washed with 10 mL of hydrochloric acid (10% w/w). Organic phase was extracted and 5 mL of an aqueous solution saturated by sodium iodide was added. The mixture was stirred, at room temperature, for 24 hours. Organic phase was extracted, washed twice by 10 mL of water, dried and evaporated under vacuum. The product was purified by successive precipitation in diethylether at −4 degree C. and isolated as a white powder (yield: 90%).

### Example 7

### Synthesis of ditetradecyl(3-trimethylarsoniopropyl)phosphonate iodide

2.9 grams of ditetradecyl(3-bromopropyl)phosphonate and 0.9 mL of trimethylarsine were heated in a sealed tube (closed under vacuum), for a week, at 45 degree C. The product was then taken up in dichloromethane and washed twice by 10 mL of hydrochloric acid at 10%. Metathesis of the counter-ion was performed as described above, and after work-up (washing, drying, evaporation) the product was purified by successive precipitation in diethylether (yield: 90%).

### Example 8

### (3-trimethylphosphonio)propylen-1,2-dioleate and (3-trimethylarsonio)propylen-1,2-dioleate

3.6 grams of 3-iodopropylen-1,2-dioleate and 10 mL of a 1M solution of trimethylphosphine in tetrahydrofuran (or 0.9 mL of trimethylarsine) were heated in sealed tube (closed under vacuum), for at least a week, at 25 degree C. Then the product was then taken up in dichloromethane, washed twice by 10 mL of hydrochloric acid at 10%, dried on MgSO₄ and evaporated under vacuum. The product was purified by successive precipitation in a diethylether/ethylacetate mixture (90/10) (quantitative yields).

### Example 9

### In Vitro Transfection of a Nucleic Acid of Interest Complexed with a Lipophilic Compound According to the Invention

A. Materials and Methods

1. Cell Lines and Plasmid DNA

For the in vitro experiments were used the CFT-1, K562 HT29 and Hela cell lines. CFT-1 cells are SV40 large T-transformed CF-tracheal cells obtained from a CF foetus after therapeutic abortion). They were grown in MEM/Ham-F-12 (50/50) medium supplemented with 10% of foetal calf serum (FCS), 0.2 mM glutamine, 100 U/ml penicillin, 100 μg/ml streptomycin and 1% fungizone. K562 cells, HT29 and Hela maintained in RPMI-1640 medium, DMEM or MEM respectively and supplemented with 10% fetal calf serum (FCS), 0.2 mM glutamine, 100 U/ml of penicillin, 100 U/ml of streptomycin and 1% fungizone. All cells were maintained in 5% CO₂ and at 37° C.

The plasmids used were pTG11033, encoding luciferase protein and pCMVLacZ, containing the LacZ gene encoding β-galactosidase under the control of the cytomegalovirus (CMV)

2. Preparation of Cationic Phosphonolipid/DNA Complexes.

Each of the cationic phosphonolipids was prepared alone or in combination with the neutral lipid DOPE or cholesterol (Sigma, Saint Quentin Fallavier, France). The phosphonolipids were formulated by mixing chloroform solutions of the different lipids in glass vials, then removing the chloroform by rotary evaporation to produce dried lipid films. Sterile pyrogen-free Dl water was then added and the vials were sealed and stored overnight at +4° C. Small unilamellar vesicles (suv) were prepared by sonicating the compounds for 10 minutes in a sonicator (Prolabo, Paris, France). To prepare the cationic phosphonolipids/DNA complexes, plasmid DNA was first diluted with sterile pyrogen-free Dl water and added to the lipid solution. The lipoplexes were kept 30 min at room temperature before being administered into animals or used for in vitro transfections.

3. In Vitro Transfection and Reporter Gene Assay.

Transfection activity of the cationic lipid/DNA complexes in vitro was assessed using CFT1 cell lines. Cells were seeded onto a 96-well tissue culture plate at 20000 per well (16 wells per lipid tested) 24 hours before transfection and incubated overnight in a humidified 5% CO₂ atmosphere at 37° C. Transfection of the cells was performed as described by Felgner et al (Proc. Natl. Acad. Sci. USA, 1987, 84, 7413) with the following modifications: appropriate amounts of the cationic lipids and the plasmid vector pTG11033 in OptiMEM were complexed and 100 μl were added to each well. After 2.5 hours at 37° C., the cells were supplemented with 200 μl of appropriate growth medium. Following a further 48 hours at 37° C., the cells were assayed for βgalactosidase expression using a chemiluminescent assay (Clontech). Assays were carried out as described by the manufacturer. The results were expressed in TRLU (Total Relative Light Units obtained for 16 wells)

4. Determination of Cell Toxicity

The relative cytotoxicity of the different lipid:DNA complexes were determined as the number of cells surviving the transfection experiment measured using a chemiluminescent assay: CYTOLITE assay (Pakard) as specified by the manufacturer. 24 hours before the assay, the cells were plated in a 96-wells plate (25000 cells per well). Cells were treated for transfection as described above and incubated for an additional 48 hours period. After this time the cytotoxicity assay was carried out as specified by the manufacture. The amount of relative light units (RLU) formed was proportional to the number of living cells. Non transfected cells were used as control. The final results were expressed in toxicity index. This toxicity index was the calculated ratio of number of living cells in the “control well” over the number of living cells in the “transfected well”. A toxicity index of 1 shows no differences between control and transfected cells implying no cytotoxicity. Cytotoxicity index increased as the toxicity of the cationic lipid tested increased

B. In Vitro Comparative Results

For a strict comparative evaluation of the efficiency of new claimed phosphonium and arsonium lipids with their yet known ammonium analogues, each preparation as described in point 2 of “Materials and methods” was targeted at each cell line stated in point 1 of “Materials and methods”, at the same time and the same conditions described in point 3 of “Materials and methods”.

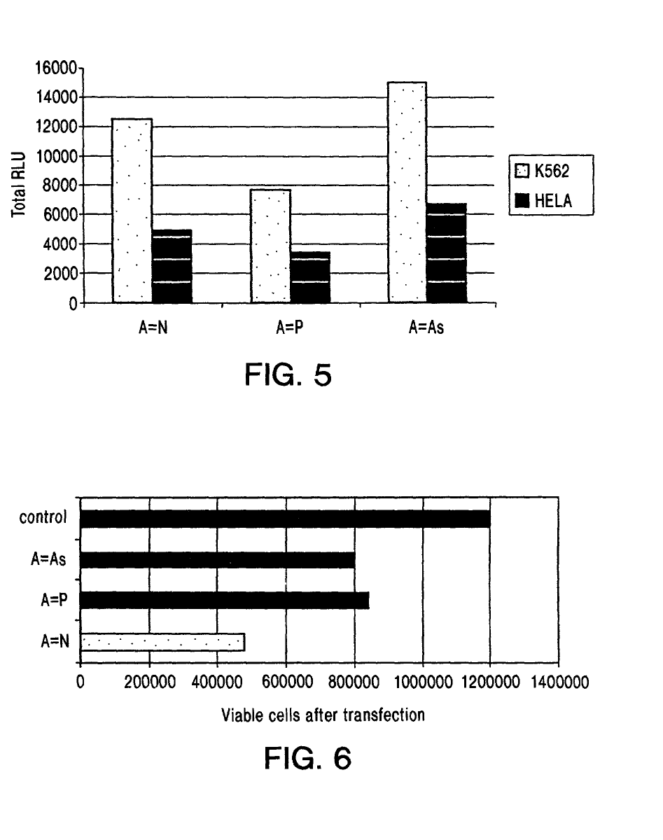

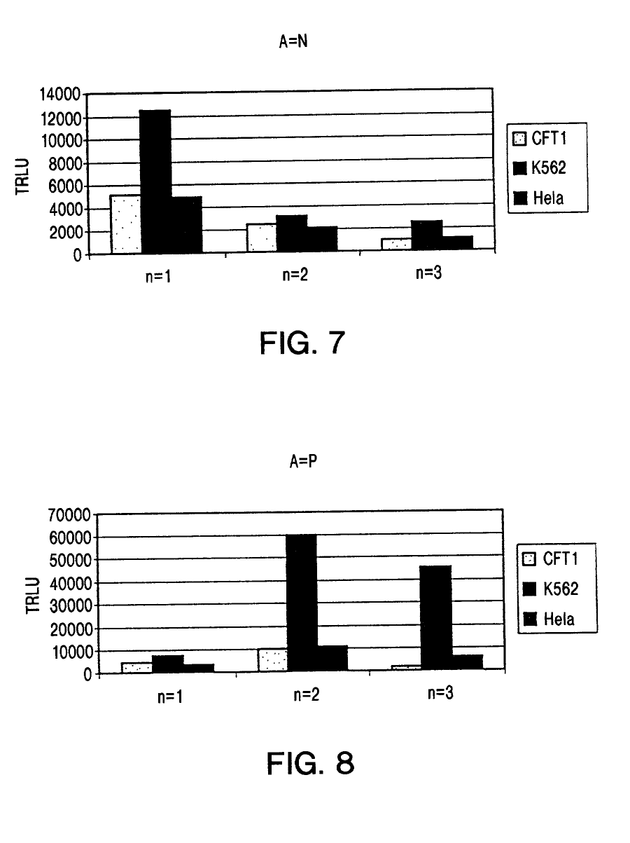

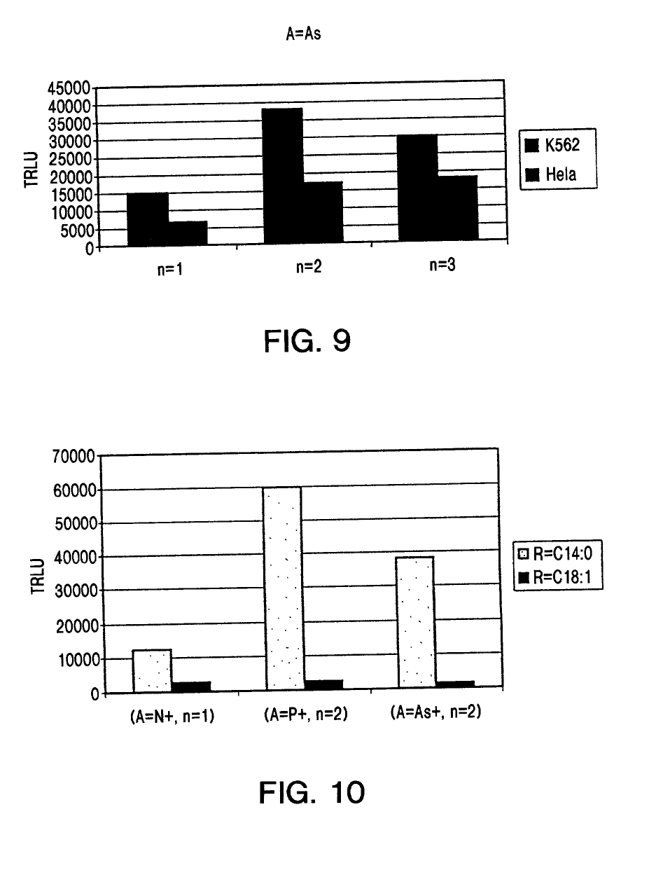

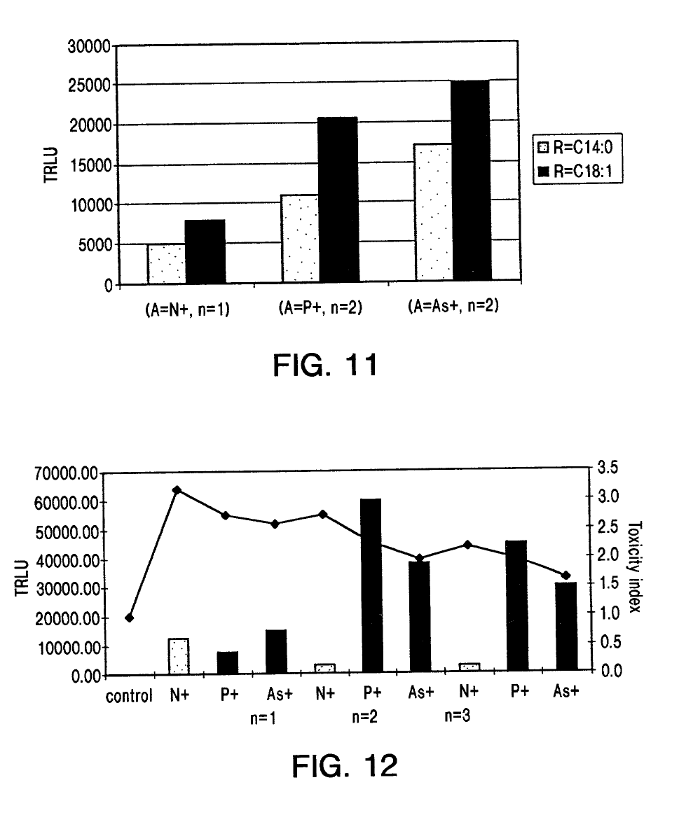

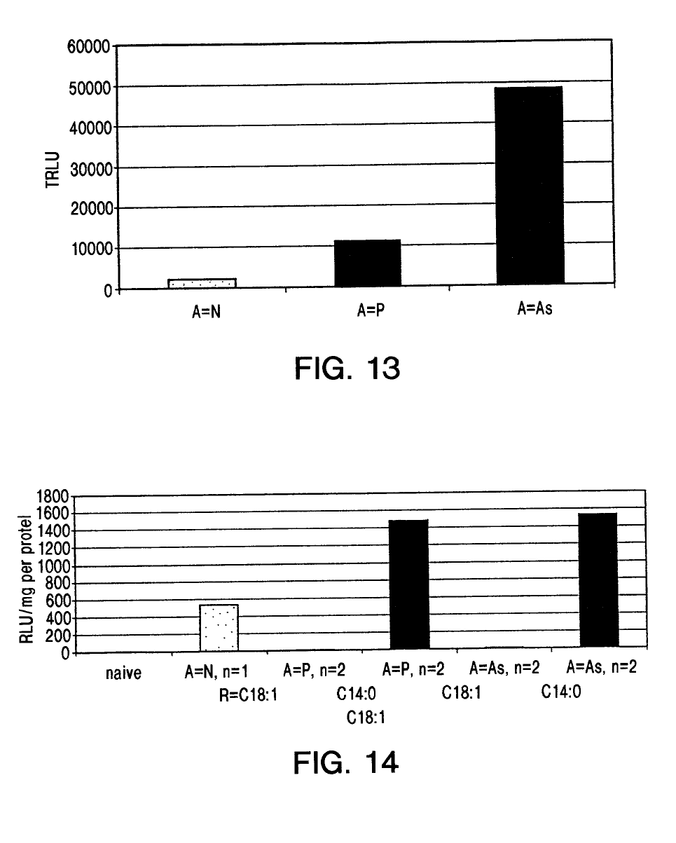

1: Comparison of the activity (on K 562 and Hela cells) (FIG. 5) and of the toxicity (on K 562 cells) (FIG. 6) of three ditetradecylphosphonates were the cation is ammonium, phosphonium and arsonium, and n=1:

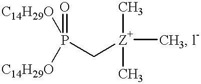

The results presented in FIGS. 5 and 6 show that lipophilic compound of the invention which contain a phosphonium or an arsonium cation are in the same range of activity as the same lipophilic compound containing an ammonium cation, but are about twice less cytotoxic on the K 562 cell line.

2: Comparison of the effect of lengthening the carbon chain between the phosphonate and the cation in a series of ditetradecylphosphonates bearing an ammonium cation (FIG. 7), a phosphonium cation (FIG. 8) or an arsonium cation (FIG. 9) on different cell lines:

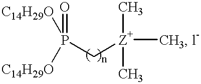

Lipophilic compounds of the structural formula above have been synthetized and contain a cation A which is either an ammonium (N), a phosposhonium (P) or an arsonium (As).

Moreover, for each cation, different chain length have been assayed, respectively n=1, 2 or 3.

Transfection of a complex between each of the compounds according to the invention as defined above with a plasmid containing an expression cassette encoding the luciferase polypeptide to cells of the CFT1, K 562 and HeLa cell lines have been performed and the results are shown in FIGS. 7, 8 and 9, respectively.

Whatever the cell line assayed, the results obtained demonstrate that compounds containing an ammonium cation have a better activity for a short chain, when n=1, whereas the compounds containing a phosphonium or an arsonium cation s were more efficient for cell transfection for longer chains (n=2 or 3).

3: Comparison of the influence of the nature of the fatty chains (C_{14:0} and C_{18:1}) on the transfection efficiency targeted at K562 (FIG. 10) and Hela (FIG. 11) cell lines.

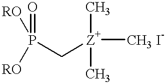

Transfection of K 562 (FIG. 10) or HeLa (FIG. 11) cell lines have been performed with the luciferase expression plasmid complexed with a lipophilic compound containing either an ammonium (N), phosphonium (P) or arsonium (As) cation and containing either a C14:0 or a C18:1 lipid moiety, wherein the first number designates the number of carbon atoms and the second number designates the number of double bonds.

For K 562 cells, the lipids with C_{14:0} chains have a good efficiency, whereas with C_{18 1}, the activity was very weak. On the contrary, the latter were more efficient in Hela cells.

4: A general comparison of the efficiency, together with a determination (as described in point 4 of “Materials and Methods”) of the cytotoxicity of several ditetradecylphosphonates on a K562 cell line (histogram 8).

From the results of FIG. 12, it is obvious that the better efficiency of phosphonium and arsonium cations (for n=2 or 3) goes together with a decreased toxicity index while going from n=1 to n=3 and from nitrogen to phosphorus then to arsenic.

5: Comparison of the efficiency of three iodides of trimethylammonium, phosphonium and arsonium propylen-1,2-dioleates (where R5 is C_{17:1} and X=I) on Hela cells (FIG. 13):

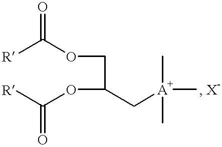

The results presented in FIG. 13 show clearly that the transfectiion efficiency is increased in the order N<P<As, with a compound containing an arsonium cation being about twenty times more efficient than the corresponding compound containing an ammonium cation.

### Example 10

### In Vivo Comparative Results:

A—Materials and Methods:

1. Intavenous Gene Delivery and Luciferase Expression in Mouse Tissues.

Cationic phosphonolipid/pTG11033 plasmid DNA complexes were delivered by a single injection of 200 μl in the tail vein of 5 week-old female BALB/c mice, each animal received 50 μg of plasmid DNA. Animals were killed 24 h after injection and mouse tissues were immediately frozen on dry ice and stored at −70° C. until examined. Luciferase activity was assayed using a chemiluminescent kit (Promega). Extraction of luciferase from mouse tissues was carried out as described previously (Thierry et al, Proc. Natl. Acad. Sci. USA, 1995, 92, 9742-9746.). The total protein concentration of the tissue extract was determined using the Bio-Rad Protein Assay. Luciferase activity of each sample was normalized to the relative light unit (RLU) per mg of extracted protein.

2. Microscopic Study of Hepatic Toxicity

Mice were sacrificed 24 hours after intravenous injection. Livers were excised and immediately fixed in “Boin fixative solution” for 24 to 48 hours. They were then processed by usual methods of paraffin embedding sections and stained with hematoxylin and eosin. Sections were examined with a photonic microscope.

B. Results

The comparative in vivo efficiency of a series of phosphonolipids bearing an ammonium, a phosphonium or an arsonium cation was achieved on mice, as described in point 4 of “Materials and methods”, each lipid being formulated alone or with DOPE or cholesterol as co-lipid, as described in point 2 of “Materials and methods” (FIG. 14).

The phosphonolipids used are those for which R1 is of formula V, R6 is an ethyl group and R5 is a lipid moiety which has the structure C18:1.

From the results of FIG. 14, it is obvious that compounds containing a phosphonium or an arsonium cation where n=2 are very efficient when formulated with cholesterol as co-lipid, the compound containing an arsonium cation and a C_{18:1} chain being about three times more efficient than the corresponding compound containing an ammonium.

What is claimed is:

1. A compound of the general formula (I) below: 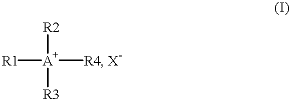
Wherein A is a phosphorus or an arsenic atom; X⁻ is an anion; and
wherein R1 is selected from the group consisting of:
a) the radical of formula (II) below: 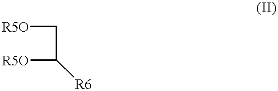
wherein R5 represents a lipid moiety and R6 is a linear or branched alkyl chain from 1 to 4 carbon atoms,
Provided that R2, R3 and R4 of formula (I) represent each a methyl group;
b) the radical of formula (III) below: 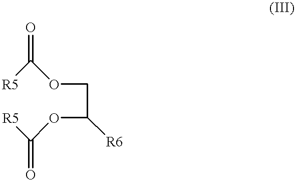
wherein R5 represents a lipid moiety and R6 is a linear or branched alkyl chain from 1 to 4 carbon atoms,
provided that R2, R3 and R4 of formula (I) represent each a methyl group;
c) the radical of formula (IV) below: 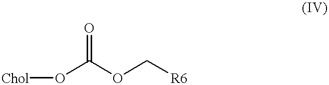
wherein Chol means a cholesteryl radical and R6 is a linear or branched alkyl chain from 1 to 4 carbon atoms,
provided that R2 and R3 of formula (I) represent each a methyl group; and
d) the radical of formula (V) below: 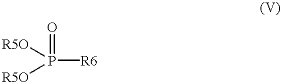
wherein R5 represents a lipid moiety and R6 is a linear or branched alkyl chain from 1 to 4 carbon atoms,
provided that R2 and R4 are alkyl chains from 1 to 4 carbon atoms; and
R3 is selected from the group consisting of:
an alkyl chain as defined for R2 and R4,
the functional group CH₂—CH₂—P⁺ (R6R7R8), wherein R6, R7 and R8 have the same meaning as R2 and R4; and
—CH₂—CO₂R9, wherein R9 has the same meaning as R2.

2. The compound of claim 1, wherein the anion X is selected from the group consisting of an halide, CF₃SO₃⁻, CF₃CO₂⁻ or HSO₄⁻.

3. The compound of claim 2, wherein the halide is selected from the group consisting of Cl⁻, Br⁻ and I⁻.

4. The compound of claim 1, wherein the R5 lipid moiety is selected from the group consisting of:
(i) an alkyl or an alkenyl chain containing from 10 to 22 carbon atoms comprising 0, 1 or 2 olefinic double bonds,
(ii) a cholesteryl derivative
(iii) a perfluoro alkyl chain from 10 to 22 carbon atoms.

5. The compound of claim 1, wherein the R5 lipid moiety is selected from the group consisting of C_{14:0}, C_{18:1}, C_{18:2}; C_{15:0}, C_{17:0}, C_{17:1}, C_{17 2}, wherein the first number designates the number of carbon atoms and the second number designates the number of double bonds.

6. The compound of claim 1, wherein R1 is of formula V and R2 and R4 represent each independently a radical selected from the group consisting of CH₃, C₂H₅, nC₃H₇, iso-C₃H₇, with n being an integer equal to 1, 2 or 3

7. The compound of claim 1 wherein R1 has the formula (II), (III) or (V), the R5 lipid moiety consists of an alkyl chain and R6 is a methyl group.

8. The compound of claim 1 wherein R1 has the formula (II), (III) or (V), the R5 lipid moiety consists of an alkenyl chain and R6 is a methyl group.

9. The compound of claim 1 wherein R1 has the formula (II), (III) or (V), the R5 lipid moiety consists of an alkyl chain and R6 is an ethyl group.

10. The compound of claim 1 wherein R1 has the formula (II), (III) or (V), the R5 lipid moiety consists of an alkenyl chain and R6 is an ethyl group.

11. The compound of claim 1 wherein R1 has the formula (II), (III) or (V), the R5 lipid moiety consists of an alkyl chain and R6 is a propyl group

12. The compound of claim 1 wherein R1 has the formula (II), (III) or (V), the R5 lipid moiety consists of an alkenyl chain and R6 is a propyl group

13. The compound of claim 1 wherein R1 has the formula (II), (III) or (V), the R5 lipid moiety consists of a cholesteryl —[C(O)N—CH₂—CH₂—O)] group and R6 is an ethyl group.

14. The compound of claim 1 wherein R1 has the formula (II), (III) or (V), the R5 lipid moiety consists of a perfluoroalkyl chain R6 is an ethyl group.

15. The compound of claim 1 wherein R1 has the formula (II), (III) or (V), the R5 lipid moiety consists of an oleoyl chain (C₁₇H₃₃C(O)O) and R6 is a propylen group.

16. A compound according to claim 1 wherein R1 has the formula (II), (III) or (V), the R5 lipid moiety consists of an oleyl chain (C₁₈H₃₅) and R6 is a −1,2 deoxyglycerol group.

17. The compound of claim 1 wherein R1 has the formula (II), (III) or (V), the R5 lipid moiety consists of a cholesteryl group and R6 is a [C(O)O—CH₂—CH₂—] group.

18. A vesicle comprising the compound according to any one of claims 1 to 17.

19. A vesicle consisting essentially of a compound according to any one of claims 1 to 17.

20. The vesicle of claim 18, which is a small unilamellar vesicle.

21. The vesicle of claim 18, which is a multilamellar vesicle.

22. A method for introducing in vitro a nucleic acid in a cell host comprising the steps of:
a) incubating said nucleic acid with a compound according to any one of claims 1 to 17 to obtain complexes formed between said nucleic acid and said compound; and
b) incubating the cell host with the complexes obtained at step a).

23. The method of claim 22, wherein the compound is under the form of unilamellar vesicles.

24. A method for introducing in vivo a nucleic acid in cells of an host organism comprising the steps of:
a) incubating said nucleic acid with a compound according to any one of claims 1 to 17 to obtain complexes formed between said nucleic acid and said compound; and
b) administering the complexes obtained at step a) to said host organism.

25. The method of claim 24, wherein the organism is a mammal.

26. A complex formed between a nucleic acid and a compound according to any one of claims 1 to 17.

27. The complex of claim 26, wherein the nucleic acid comprises a polynucleotide encoding a polypeptide.

28. The complex of claim 26, wherein the nucleic acid comprises a polynucleotide which encodes an antisense polynucleotide.

29. The complex of claim 26, wherein the polynucleotide encoding a polypeptide is operably linked to a regulatory sequence.

30. A composition comprising a complex according to any one of claims 26 to 29.

31. A pharmaceutical composition comprising a complex according to any one of claims 26 to 29.
